(19) United States
(12) Patent Application Publication
(10) Pub. No.: US 2016/0038099 A1
(43) Pub. Date: Feb. 11, 2016

(54) MAGNETIC RESONANCE IMAGING APPARATUS, MEDICAL INFORMATION PROCESSING DEVICE, AND PATIENT INFORMATION DISPLAY METHOD

(71) Applicants: KABUSHIKI KAISHA TOSHIBA, Tokyo (JP); TOSHIBA MEDICAL SYSTEMS CORPORATION, Otawara-Shi (JP)

(72) Inventors: Tsutomu IGARASHI, Otawara (JP); Junichiro ARAOKA, Nasushiobara (JP)

(21) Appl. No.: 14/920,214

(22) Filed: Oct. 22, 2015

Related U.S. Application Data
(63) Continuation of application No. PCT/JP2014/060147, filed on Apr. 8, 2014.

(30) Foreign Application Priority Data
Apr. 26, 2013 (JP) 2013/094535

(51) Int. Cl. A61B 5/00; G08B 21/18; G06T 11/60; G06T 11/00; A61B 5/055; G01R 33/54

(52) U.S. Cl. CPC A61B 5/7435; A61B 5/055; A61B 5/4872; A61B 5/743; A61B 5/746; A61B 5/7475; G01R 33/546; G06T 11/60; G06T 11/001; G08B 21/182

(57) ABSTRACT

In one embodiment, a magnetic resonance imaging apparatus includes an input device by which patient information including at least height and weight is inputted; a display; and processing circuitry configured to calculate a BMI (Body Mass Index) indicated by weight (kg)/{height (m)}² based on the height and weight, determine whether the BMI is within a range of a predetermined threshold value or not; and cause the display to display an alarm when the BMI is not within the range of a predetermined threshold value.

### CROSS-REFERENCE TO RELATED APPLICATIONS

This application is a Continuation application of No. PCT/JP2014/60147 filed on Apr. 8, 2014, and the PCT application is based upon and claims the benefit of priority from Japanese Patent Application No. 2013-94535, filed on Apr. 26, 2013, the entire contents of which are incorporated herein by reference.

### FIELD

Embodiments described herein relate generally to a magnetic resonance imaging apparatus, a medical information processing device, and a patient information display method.

### BACKGROUND

A magnetic resonance imaging apparatus is an imaging apparatus which excites nuclear spin of a patient placed in a static magnetic field with an RF (Radio Frequency) signal having the Larmor frequency and reconstructs an image based on magnetic resonance signals emitted from the patient due to the excitation.

In imaging with the use of a magnetic resonance imaging apparatus, high-frequency (RF) signals are applied to a patient in order to obtain magnetic resonance signals. Application of high-frequency signals heats up the patient and increases body temperature of the patient. For the above reason, SAR (Specific Absorption Ratio) is defined as energy absorbed per unit mass of a patient from the perspective of safety. In addition, upper limit values of SAR i.e. safety reference values of SAR are defined in IEC (International Electrotechnical Commission) standards (IEC60601-2-33).

More specifically, SAR (its unit is W/kg) is defined as energy of RF signal(s) absorbed by one kilogram of biological tissue. An upper limit value of time average SAR in arbitrary ten seconds and an upper limit value of time average SAR in the last six minutes are defined for each imaging part such as the whole body, the head part, etc. Hereinafter, the above time average SAR in arbitrary ten seconds is simply referred to as 10-second average SAR, and the time average SAR in the last six minutes is simply referred to as 6-minute average SAR.

Accordingly, imaging is performed in such a manner that the 10-second average SAR and 6-minute average SAR do not exceed the defined upper values of SAR.

Since SAR is defined as energy absorbed per unit mass of a patient, weight information of a patient is necessary for calculating the 10-second average SAR, 6-minute average SAR, and/or upper limit values of SAR. In addition, when a part of a patient is imaged, height information of the patient is sometimes used in addition to the weight information in order to estimate the partial weight of the imaging part.

As mentioned above, height and weight of a patient is important information for calculating SAR. Therefore, an inspection engineer generally inputs height and weight of a patient by using an input device such as a keyboard. from a console of a magnetic resonance imaging apparatus, before imaging with the use of the magnetic resonance imaging apparatus is started.

However, occurrence of a human error cannot be completely prevented because input of height and weight is performed by manual operation.

There is a method of prompting an operator to confirm height and weight by redisplaying the inputted height and weight on a screen of a console. However, since an operator such as an inspection engineer is convinced in the first place that correct values has been inputted, the operator does not so seriously confirm the redisplayed height and weight in many cases. Therefore, to simply redisplay the inputted values has little effect on eliminating a human error.

Although adding a function of measuring weight of a patient to a bed of a magnetic resonance imaging apparatus is possible as another method, this method makes the apparatus more complicated and increases manufacturing cost. Although weight of surface coils set on a patient must be subtracted in this method, the total weight of surface coils is different depending on their types and number. Thus, this method makes operation of a magnetic resonance imaging apparatus more complicated because manual input of information on the total weight of surface coils is further needed.

For the above reason, a magnetic resonance imaging apparatus and a medical information processing device which are capable of infallibly prevent incorrect input of height and weight by an easy method have been desired.

### BRIEF DESCRIPTION OF THE DRAWINGS

FIG. 1 is a block diagram showing an example of overall configuration of a magnetic resonance imaging apparatus of one embodiment;

FIG. 2 is a block diagram showing an example of various types of apparatuses and devices interconnected via a network in a hospital;

FIG. 3 is a chart explaining an example in which height and weight are mistakenly inputted;

FIG. 4 is a functional block diagram mainly showing functions relevant to prevention of erroneous input of height and weight, out of all the functions of a console of the magnetic resonance imaging apparatus;

FIG. 5 is a functional block diagram mainly showing functions relevant to prevention of erroneous input of height and weight, out of all the functions of a medical information processing device connected to a network in a hospital;

FIG. 6 is a flowchart showing an example of processing of preventing erroneous input of height and weight;

FIG. 7 is a chart showing an example of a display aspect of height, weight, and relationship between a calculated BMI and a threshold value;

FIG. 8 is a chart showing an example of an alarm displayed on an input screen of the console;

FIG. 9 is a chart showing an example of an icon displayed on the input screen when height and weight are correctly inputted; and

FIG. 10 is a chart showing an example of an icon displayed on the input screen when height and weight are erroneously inputted.

### DETAILED DESCRIPTION

An magnetic resonance imaging apparatus of one embodiment includes: an input device by which patient information including at least height and weight is inputted; a display; and processing circuitry configured to calculate a BMI (Body Mass Index) indicated by weight (kg)/{height (m)}² based on the height and weight, determine whether the BMI is within a range of a predetermined threshold value or not; and cause the display to display an alarm when the BMI is not within the range of a predetermined threshold value.

Hereinafter, embodiments of the present disclosure will be explained with reference to the accompanying drawings.

(1) Overall Configuration

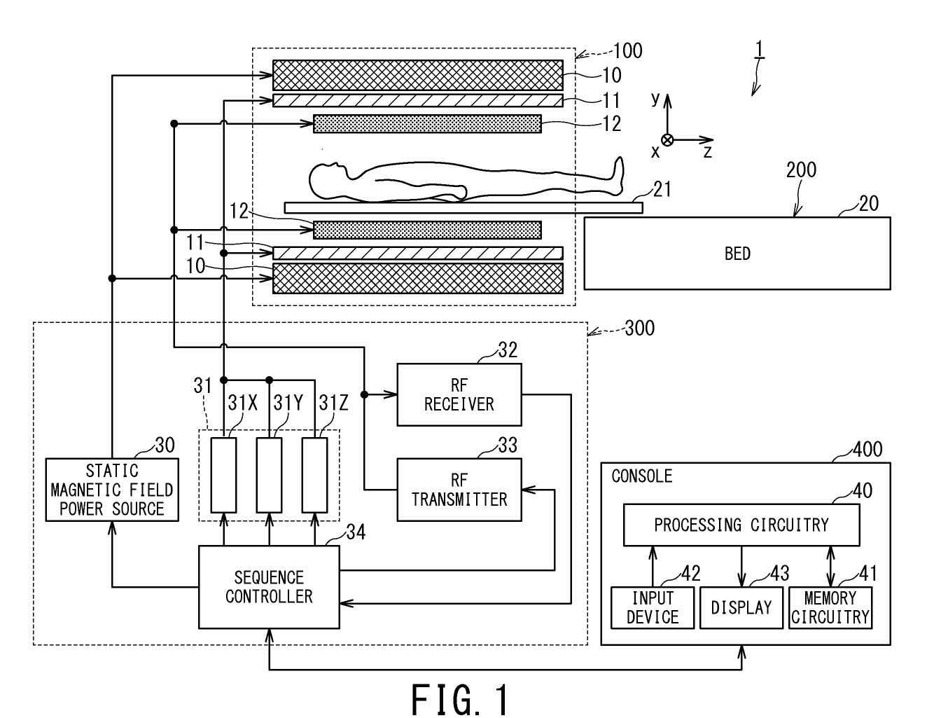

FIG. 1 is a block diagram showing an example of overall configuration of the magnetic resonance imaging apparatus 1 of the present embodiment. The magnetic resonance imaging apparatus 1 of the present embodiment includes a gantry 100, a bed 200, control cabinet components 300, and a console 400.

The gantry 100 includes a static magnetic field magnet 10, a gradient coil 11, and an RF coil 12. and these components are housed in a cylindrical case. The bed 200 includes a bed body 20 and a table 21.

The control cabinet components 300 include a static magnetic field power source 30, gradient coil power sources 31, an RF receiver 32, an RF transmitter 33, and a sequence controller 34. The gradient coil power sources 31 includes an X axis gradient coil power source 31x, a Y axis gradient coil power source 31y, and a Z axis gradient coil power source 31z. In addition, the console 400 is configured as a computer including processing circuitry 40, memory circuitry 41, an input device 42, and a display 43.

The static magnetic field magnet 10 of the gantry 100 is substantially in the form of a cylinder. The static magnetic field magnet 10 generates a static magnetic field inside the bore, which is an internal space of the cylindrical structure thereof and functions as an imaging space for an object such as a patient.

The static magnetic field magnet 10 includes a superconductive coil inside and this superconductive coil is cooled down to an extremely low temperature by liquid helium. The static magnetic field magnet 10 generates the static magnetic field by supplying the superconductive coil with the electric current provided from the static magnetic field power source 30 in an excitation mode. Afterward, when the static magnetic field magnet 10 shifts to a permanent current mode, the static magnetic field power source 30 is separated. Once it enters the permanent current mode, the static magnetic field magnet 10 continues to generate a strong static magnetic field for a long time, for example, over one year. Incidentally, the static magnetic field magnet 10 may be configured as a permanent magnet.

The gradient coil 11 is also substantially in the form of a cylinder and is fixed to the inside of the static magnetic field magnet 10. This gradient coil 11 applies gradient magnetic fields to the imaging space in the respective directions of the X axis, the Y axis, and the Z axis, with the electric currents supplied from the above-described gradient coil power sources 31x, 31y, and 31z.

The bed body 20 of the bed 200 can move the table 21 in the upward and downward directions, and moves the object loaded on the table 21 to a predetermined height before imaging. Afterward, at the time of imaging, the bed body 20 moves the table 21 in the horizontal direction so as to move the object inside the bore.

The RF coil 12 arranged inside the gradient coil 11 is also called a whole body coil. The RF coil 12 is also substantially in the form of a cylinder and is fixed to the inside of the gradient coil 11. The RF coil 12 applies RF pulses transmitted from the RF transmitter 33 to the object and receives the magnetic resonance signals emitted from the object due to excitation of hydrogen atoms.

The RF transmitter 33 transmits RF pulses to the RF coil 12 based on a command from the sequence controller 34. On the other hand, the RF receiver 32 receives the magnetic resonance signals received by the RF coil 12 and transmits k-space data obtained by digitizing the received magnetic resonance signals to the sequence controller 34.

The sequence controller 34 performs a scan of the object under the control of the console 400, by driving each of the gradient coil power sources 31, the RF transmitter 33, and the RF receiver 32. When the sequence controller 34 receives the k-space data from the RF receiver 32 by performing the scan, the sequence controller 34 transmits the k-space data to the console 400.

The console 400 controls the entirety of the magnetic resonance imaging apparatus 1. Specifically, the input device 42 includes an input circuit and input tools such as a keyboard, a mouse. The console 400 receives imaging conditions, various types of information, and commands inputted by an operator such as an inspection engineer, via the input device 42 such as the mouse, the keyboard.

Then, the processing circuitry 40 makes the sequence controller 34 perform a scan based on the inputted imaging conditions, and reconstructs images based on the k-space data transmitted from the sequence controller 34. The reconstructed images are displayed on the display 43 and/or stored in the memory circuitry 41.

In the magnetic resonance imaging apparatus 1 of the present embodiment, the above-described calculation of SAR is performed by the console 400. Specifically, patient information such as height and weight of the patient is inputted by an inspection engineer via the input device 42 of the console 400 such as the keyboard. Thereby, an input circuit (not shown) in the input device 42 receives and temporarily stores the inputted patient information, and transmits the inputted contents to the processing circuitry 40.

The processing circuitry 40 of the console 400 calculates an upper limit value of SAR based on the inputted information such as height and weight. In addition, when imaging conditions such as a pulse sequence are set via the input device 42, the processing circuitry 40 performs predictive calculation of SAR of imaging to be performed based on various factors of RF pulses corresponding to the imaging conditions and the height and weight of the patient. In this case, the processing circuitry 40 determines whether the SAR value calculated based on the set imaging conditions is within the upper limit value of SAR or not.

Furthermore, the processing circuitry 40 measures actual RF power after start of imaging and calculates an actual measured value of SAR based on the measured RF power value and the height and weight of the patient. When an actual measured value of SAR is likely to exceed the upper limit value of SAR, the processing circuitry 40 outputs such a control signal that imaging operation is immediately and safely stopped before SAR exceeds the upper limit, to the sequence controller 34.

Although the height and weight of the patient can be inputted via the console 400 of the magnetic resonance imaging apparatus 1, they are sometimes inputted via a medical information processing apparatus 501 such as an RIS (Radiology Information System) installed at another place in a hospital.

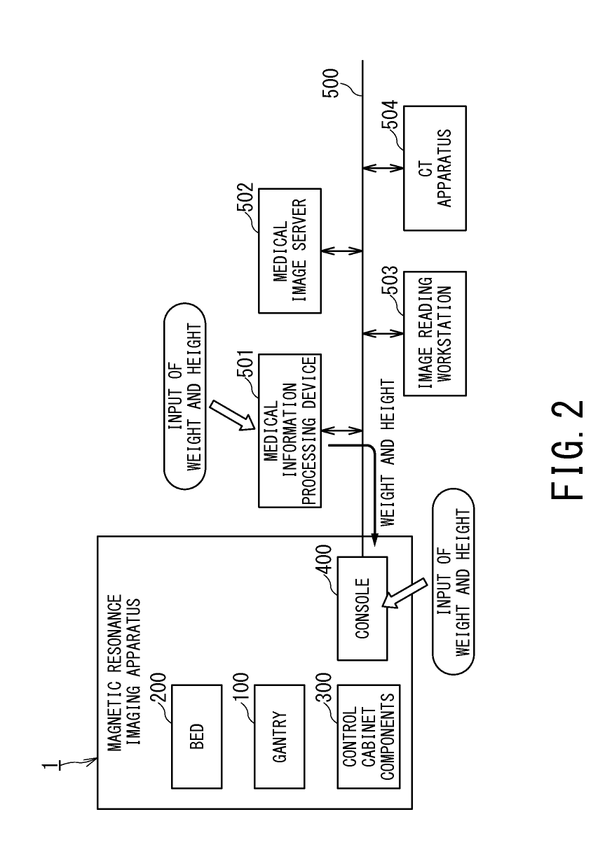

FIG. 2 is a block diagram showing an example of various types of apparatuses and devices interconnected via the network 500 in a hospital. The magnetic resonance imaging apparatus 1 and other modalities such as a CT apparatus 504 are connected to the network 500. Moreover, a medical image server 502 which stores medical image data imaged by each modality, an image reading workstation 503 for reading images, the medical information processing device 501, etc. are connected to the network 500.

Patient information including height and weight is inputted to the medical information processing device 501 and the medical information processing device 501 generates an imaging order including examination contents for imaging the patient by using a modality such as the magnetic resonance imaging apparatus 1. When magnetic resonance imaging is performed, this imaging order is transmitted to the console 400 of the magnetic resonance imaging apparatus 1.

As mentioned above, information on height and weight of a patient is used for calculation of SAR. Thus, there is a possibility that a patient is exposed to danger if incorrect information is inputted. For example, if a value larger than the actual weight of a certain patient is inputted as the weight of the patient due to erroneous operation by an inspection engineer etc., the upper limit value of SAR is calculated as an incorrect value larger than the correct value. As a result, possibility of applying RF power higher than the correct upper limit value which must not be originally exceeded to a patient is generated.

By contrast, if a value smaller than the actual weight of a certain patient is inputted as the weight of the patient, the upper limit value of SAR is calculated as an incorrect value smaller than the correct value. As a result, RF power is restricted by a value smaller than the value which can be originally outputted, and images of low SNR (signal to noise ratio) are generated.

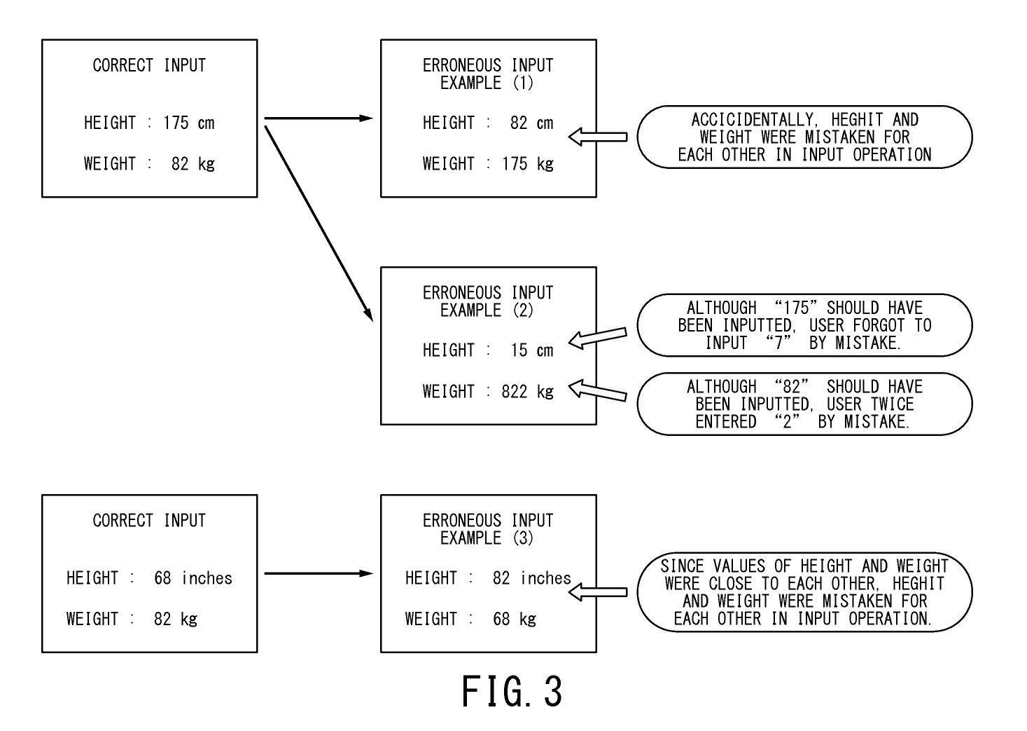

FIG. 3 is a chart explaining an example in which height and weight are mistakenly inputted. In this example, it is assumed that correct height and weight to be originally inputted are 175 cm for height and 82 kg for weight.

The erroneous input example (1) is a case where height and weight are accidentally mistaken for each other in input operation. As a result, height is erroneously inputted as 82 cm and weight is erroneously inputted as 175 kg in this case.

The erroneous input example (2) is a case where 175 and 82 should be inputted as the respective correct values of height and weight but the middle letter “7” of the height is not entered by mistake such as mistouch of a keyboard etc. and the final letter “2” of the weight is twice entered by mistake. As a result, height is erroneously inputted as 15 cm and weight is erroneously inputted as 822 kg.

In each of the erroneous input examples (1) and (2), an incorrect weight value larger than the correct value is inputted. As a result, the upper limit value of SAR is calculated as a value considerably larger than the originally correct value. Therefore, even if large RF power is applied to a patient because of some reasons and the actual SAR exceeds the correct upper limit value of SAR, application of RF power cannot be immediately stopped. In this case, the patient is exposed to danger.

The erroneous input example (3) is a case where height is entered as in units of inch in the magnetic resonance imaging apparatus 1 configured as the American specification. When height is entered as in units of inch and weight is entered as in units of kilogram, the value of height becomes comparatively close to the value of weight. Thus, there is a high possibility that height and weight are mistaken for each other by an operator in input operation and the operator does not notice it.

An operator, such as an inspection engineer, who has entered values is convinced that he or she has entered correct values. Therefore, once the operator enters an incorrect value by mistake as mentioned above, the operator can hardly discover his or her erroneous input. Thus, it was conventionally difficult to completely eliminate even such a human error that is likely to be easily discovered at first glance.

Each of the magnetic resonance imaging apparatus 1 and the medical information processing device 501 of the present embodiment resolves the above-described conventional problem and can effectively prevent erroneous input of height and weight by an easy method. Hereinafter, details of the method of preventing erroneous input will be explained.

(2) Prevention of Erroneous Input of Height and Weight

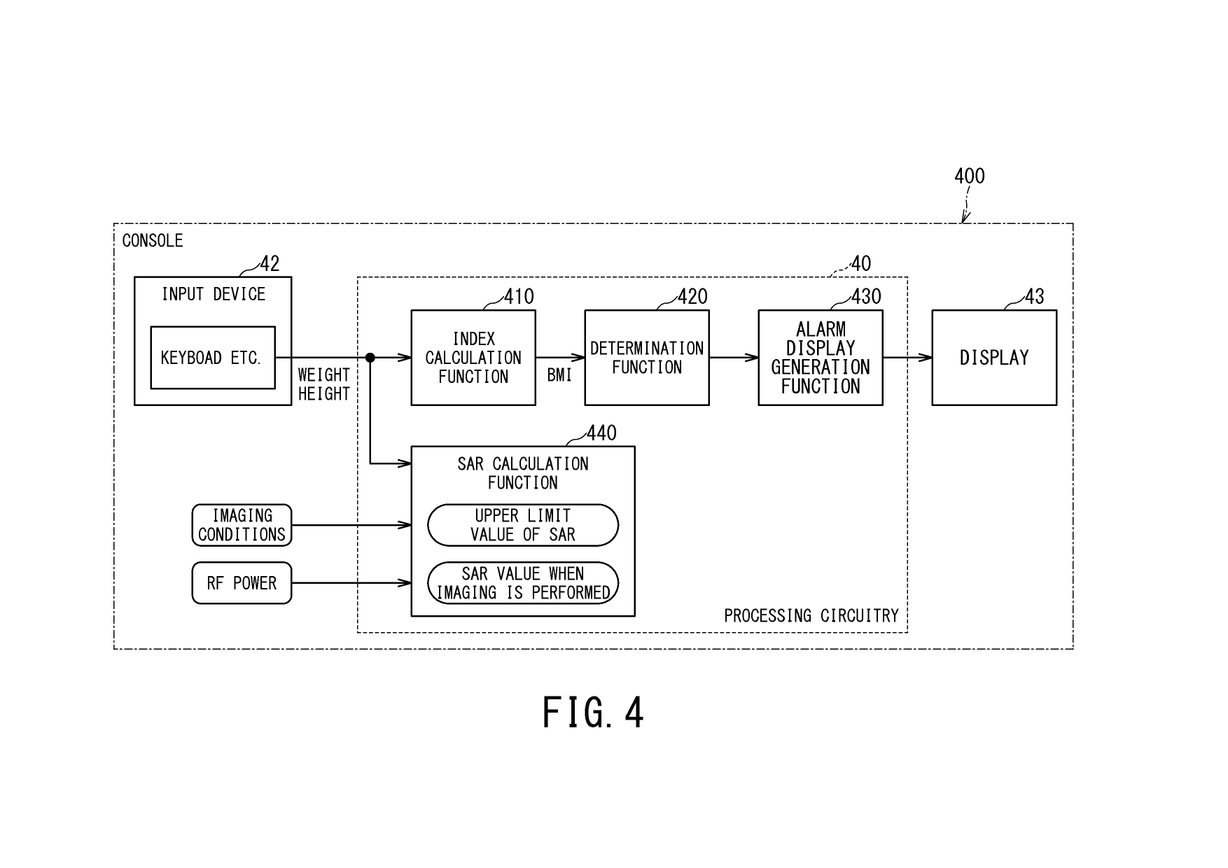

FIG. 4 is a functional block diagram mainly showing functions relevant to prevention of erroneous input of height and weight, out of all the functions of the console 400 of the magnetic resonance imaging apparatus 1.

The processing circuitry 40 of the console 400 includes, for example, structure for computational processing such as a processor etc. The processing circuitry 40 implements various functions such as an index calculation function 410, a determination function 420, an alarm display generation function 430, an SAR calculation function 440, etc.

The patient information including at least height and weight is inputted by a user's operation via the input device 42 to the console 400. The index calculation function 410 calculates a BMI (Body Mass Index) value indicated by weight (kg)/{height (meter)}², based on the inputted height and weight.

The determination function 420 of the processing circuitry 40 determines whether the calculated BMI value is within the range of predetermined (upper and lower) threshold values. When the calculated BMI value is determined to be out of the range of the predetermined threshold values, the display 43 displays an alarm indicative of this determination result. The alarm display generation function 430 generates display data for causing the display 43 to display the above-described alarm.

Meanwhile, the SAR calculation function 440 of the processing circuitry 40 calculates an upper limit value of SAR based on the inputted information of height and weight. In addition, in the stage of setting imaging conditions, the SAR calculation function 440 predicts SAR based on the set imaging conditions and the inputted information of height and weight, and the determination function 420 determines whether or not the SAR predicted value is within the range of the upper limit value of SAR and under. When it is determined that the SAR predicted value exceeds the upper limit value of SAR, imaging conditions are set again.

Moreover, after start of actual imaging operation, RF power is measured, an actual measured value of SAR is calculated based on the measured RF power and the inputted height and weight, and the imaging operation is immediately and safely stopped if the actual measured value of SAR is likely to exceed the upper limit value of SAR.

In the above configuration, at least one processor of the processing circuitry 40 implements the function of each of the index calculation function 410, the determination function 420, the alarm display generation function 430, and the SAR calculation function 440, by executing a predetermined program stored in the memory circuitry 41. Additionally or alternatively, the programs executed by the processor of the processing circuitry 40 in order to implement the above functions may be directly stored in a circuit in the processor itself.

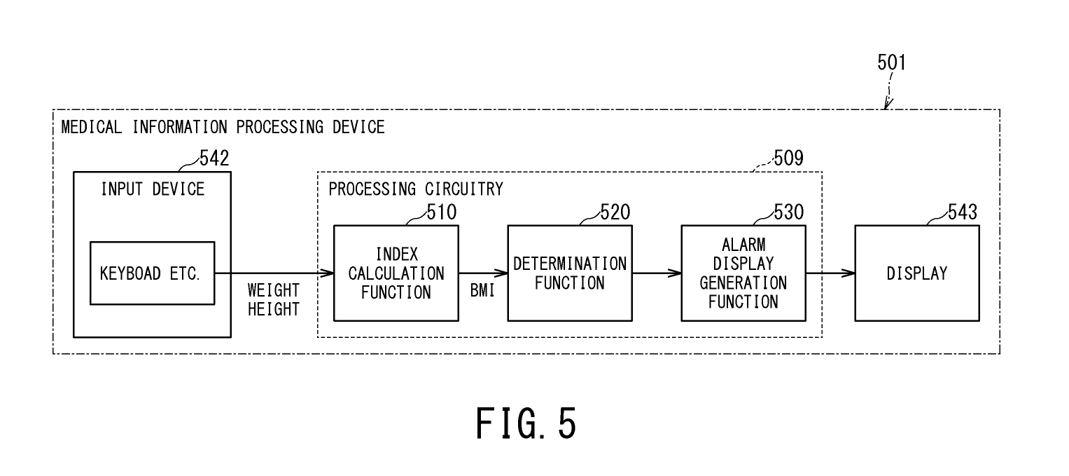

FIG. 5 is a functional block diagram mainly showing the similar functions relevant to prevention of erroneous input of height and weight, out of all the functions of the medical information processing device 501 such as an RIS connected to the network 500 in a hospital.

Similar to the console 400 of the magnetic resonance imaging apparatus 1, the medical information processing device 501 includes processing circuitry 509, a display 543 having a display panel, and an input device 542 having a keyboard, a mouse, etc.

The processing circuitry 509 includes, for example, structure for computational processing such as a processor. The processing circuitry 509 implements various functions such as an index calculation function 510, a determination function 520, an alarm display generation function 530. These functions are substantially the same as the index calculation function 410, the determination function 420, and the alarm display generation function 430 of the console 400. In addition, the functions of the input device 542 and the display 543 are the same as the input device 42 and the display 43 of the console 400. Thus, duplicate explanation is omitted. Note that the medical information processing device 501 does not have a function corresponding to the SAR calculation function 440 of the processing circuitry 40 of the magnetic resonance imaging apparatus 1.

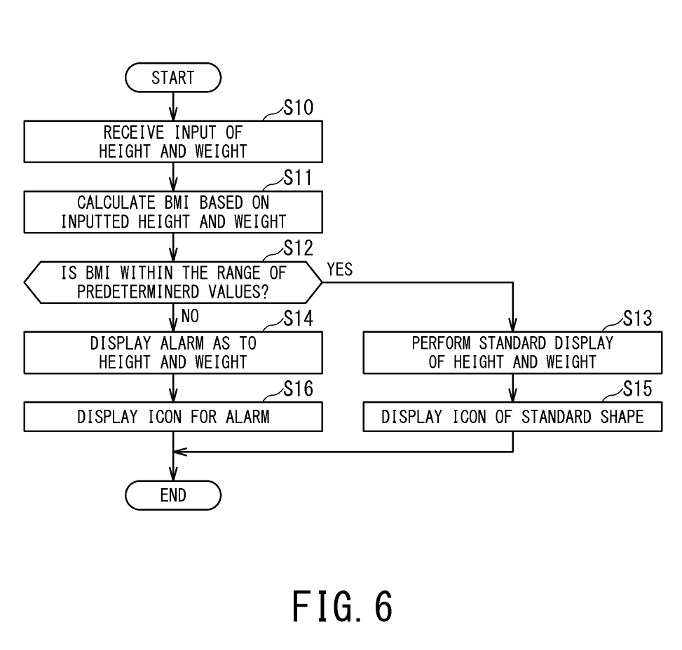

FIG. 6 is a flowchart showing an example of processing of preventing erroneous input of height and weight, out of various types of processing performed by the console 400 of the magnetic resonance imaging apparatus 1 and/or the medical information processing device 501 connected to the network 500. Hereinafter, the operation of preventing erroneous input pf the present embodiment will be explained in more detail in accordance with the step numbers in the flowchart.

In the step S10 of FIG. 6, the input device 42 of the magnetic resonance imaging apparatus 1 or the input device 542 of the medical information processing device 501 receives values of height and weight inputted by an operator. When height and weight are inputted from the magnetic resonance imaging apparatus 1, the index calculation function 410 of the processing circuitry 40 calculates a BMI value as an index for determining erroneous input based on the inputted height and weight in the step S11. On the other hand, when height and weight are inputted from the medical information processing device 501, the index calculation function 510 of the processing circuitry 509 calculates a BMI value in the way similar to the above manner. BMI is calculated by the following formula.

BMI=weight(kg)/{height(meter)}²

Since height is entered in units of [cm] in general, unit conversion from [centimeter] into [meter] is performed before calculating BMI by using the above formula. In addition, when the magnetic resonance imaging apparatus 1 is configured as the American specification etc. and its input unit for height is [inch], unit conversion from [inch] into [meter] is performed in the processing circuitry 40 of the magnetic resonance imaging apparatus 1 before calculating BMI by using the above formula.

In the step S12, determination, as to whether the calculated BMI is within the range of the predetermined (upper and lower) threshold values or not, is performed. When BMI is calculated by the magnetic resonance imaging apparatus 1, this determination is performed by the processing circuitry 40 of the magnetic resonance imaging apparatus 1. By contrast, when BMI is calculated by the medical information processing device 501, this determination is performed by the determination function 520 of the processing circuitry 509 of the medical information processing device 501.

Afterward, when the calculated BMI value is within the range of the predetermined (upper and lower) threshold values according to the determination result, height and weight are displayed by a standard aspect in the step S13. On the other hand, when the calculated BMI value is out of the range of the predetermined threshold values, the alarm of height and weight is displayed by an aspect different from the above-described standard aspect in the step S14. When the above-described determination is performed in the magnetic resonance imaging apparatus 1, the display in the step S13 and S14 is performed by the display 43 of the magnetic resonance imaging apparatus 1. On the other hand, when the above-described determination is performed in the medical information processing device 501, the display in the step S13 and S14 is performed by the display 543 of the medical information processing device 501.

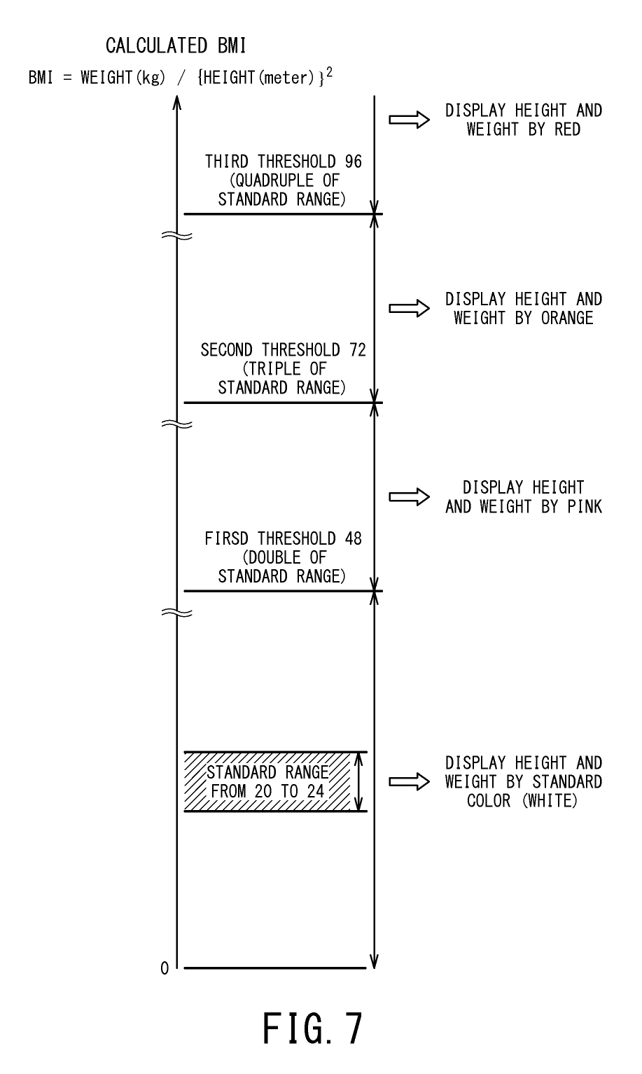

FIG. 7 is a chart showing an example of a display aspect of height, weight, and relationship between a calculated BMI and a threshold value. Although a standard value of BMI is slightly different depending on each country and each race, the range from 20 to 24 is assumed to be the standard range of BMI in Japan.

Considering the above assumption, for example, “48” as double of the upper limit value of the standard range of BMI is defined as the first threshold value. Accordingly, when the calculated BMI value is lower than the first threshold value (i.e. 48), height and weight are displayed with a standard color, for example, white. On the other hand, when the calculated BMI value is equal to or higher than 48 as the first threshold value, the alarm is displayed. As an example of the alarm, height and weight are displayed with a color different from the above-described standard color (white) like red etc. This example is a two-stage determination system based on one threshold value (or one threshold value range).

Depending on a specified region or a medical specialty, it is possible that many obese patients come to a hospital as an example. In such a case, the number of case where the calculated BMI value exceeds the threshold value increases and an inspection engineer gets used to the alarm color (red in the above-described example) of the two-stage determination system. Thus, in the two-stage determination system based on one threshold value, there is a possibility that meaning and effect of the alarm become weaker.

For the above reason, aspects of the alarm display may be changed in a multistage manner or in a continuous manner instead of the above-described two-stage determination system. In this case, the determination function 420 of the processing circuitry 40 or the determination function 520 of the processing circuitry 509 further determines degree of deviation between the calculated BMI value and the above-described threshold value (or the range between upper and lower threshold values). The displays 43 and 543 display the multistage alarm in accordance with the degree of deviation or continuously change the aspect of the alarm in accordance with the degree of deviation.

For example, “72” as triple of the upper limit value 24 of the standard range of BMI is defined as the second threshold value, and “96” as quadruple of the upper limit value 24 is defined as the third threshold value as shown in FIG. 7. Then, the multistage alarm may be displayed in the following manner as an example: (a) when the calculated BMI value is lower than the first threshold value (48), height and weight are displayed by white, (b) when the calculated BMI value is not less than the first threshold value (48) and is less than the second threshold value (72), height and weight are displayed by pink, (c) when the calculated BMI value is not less than the second threshold value (72) and is less than the third threshold value (96), height and weight are displayed by orange, and (d) when the calculated BMI value is not less than the third threshold value (96), height and weight are displayed by red.

In addition, as to display of height and weight, the displays 43 and 543 may continuously change chroma and hue of display color of height and weight in accordance with (a) the deviation between the calculated BMI value and the above-described threshold value or (b) the deviation between the calculated BMI value and the upper limit value of the standard range of BMI.

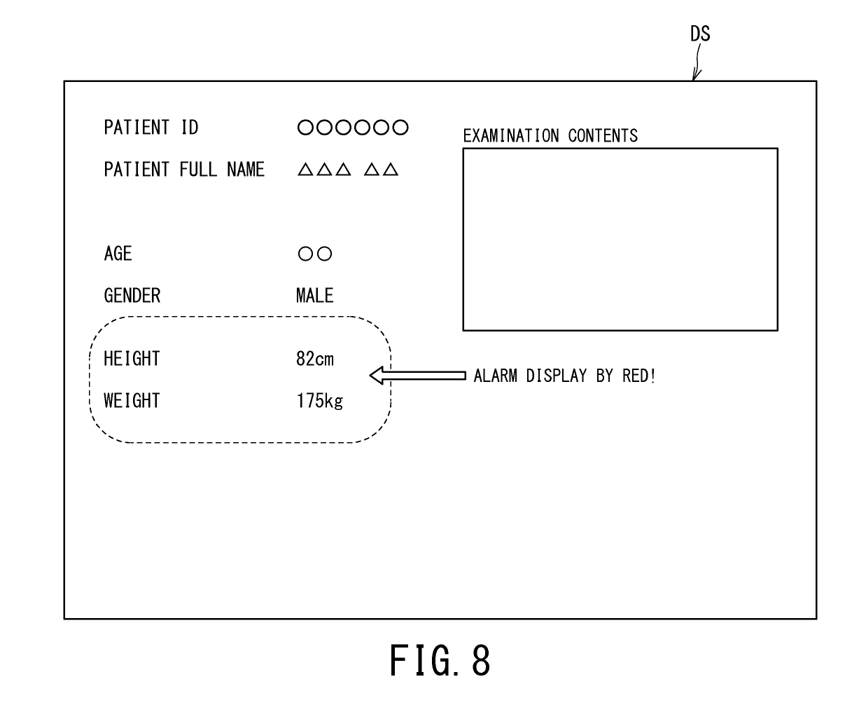

FIG. 8 is a chart showing an example of the input screen DS for entering patient information in the display 43 of the console 400 (or the display 543 of the medical information processing device 501).

When patient ID, a full name of a patient, age, gender, examination contents are sequentially entered, these contents are displayed on the input screen DS one after another. By contrast, as to height and weight, when values of these parameters are entered, the BMI value is immediately calculated and the above-described determination based on each threshold value is performed. Afterward, display color of height and weight is determined based on the determination result and height and weight are displayed by the determined color.

FIG. 8 is an example of erroneous input in which height and weight are mistaken for each other. In this case, BMI is calculated as an abnormally large value of approximately 260 and it exceeds the third threshold value shown in FIG. 7. Therefore, height and weight are displayed by red as the alarm.

In the above explanation, the alarm display is performed by displaying letters and numbers indicative of height and weight with color different from normal color. However, aspects of the alarm display are not limited to change in color.

For example, the displays 43 and 543 may perform the alarm display by changing size and/or thickness of characters (including number and symbol) indicative of height and weight from normal size and/or thickness. Additionally or alternatively, the displays 43 and 543 may perform the alarm display by appropriately combining change in color, size, and thickness of characters indicative of height and weight. In other words, the displays 43 and 543 may perform the alarm display by displaying the entered values of height and weight with such characters that at least one of color, size, and thickness is changed from a case where BMI is within the range of the predetermined threshold values.

Moreover, it is preferable that the input device 42 of the magnetic resonance imaging apparatus 1 and the input device 542 of the medical information processing device 501 are configured to be able to change at least either of the following parameters by a user's operation. The above-described parameters to be preferably changed by an operator are (a) the respective threshold values relevant to the alarm display (the first to fourth threshold values etc.), (b) character color used for the alarm display, (c) character size used for the alarm display, and (d) character thickness used for the alarm display.

In other words, it is preferable to configure the input devices 42 and 542 so that aspects of the alarm display can be customized by a user.

As shown in the step S15 and S16 in FIG. 6, the displays 43, 543 may further display an icon similar to human body shape on the input screen DS in addition to displaying values of height and weight as the alarm. This is so that attention of an operator is further drawn.

For example, the displays 43 and 543 display the icon similar to human body shape in addition to display of height and weight. When the calculated BMI value is out of the range of the predetermined threshold values, the displays 43 and 543 can draw attention of an operator by transforming the icon shape based on the BMI value and displaying the transformed icon.

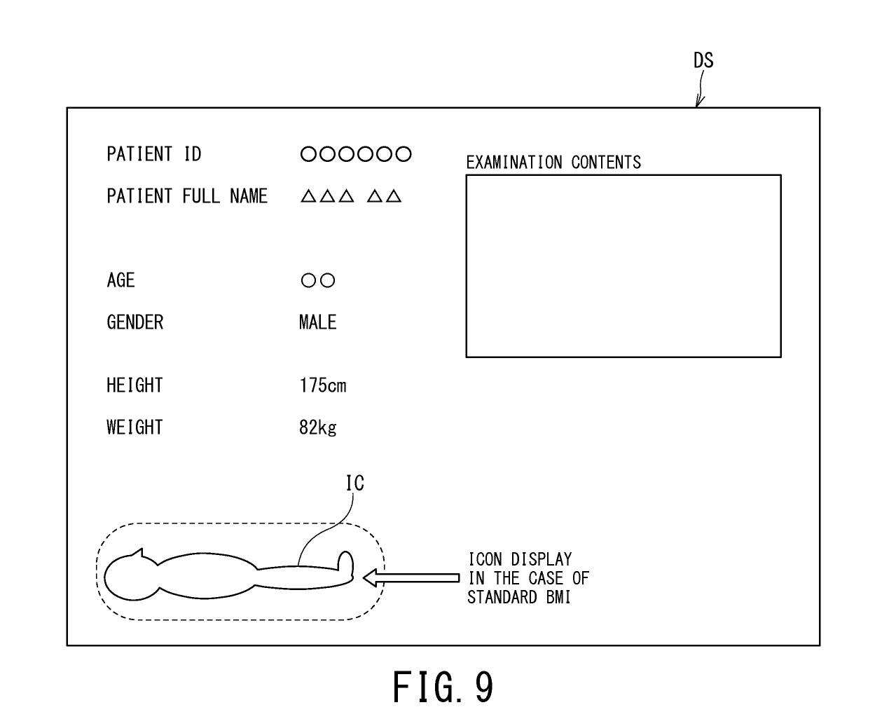

FIG. 9 is a chart showing an example of an icon IC displayed on the input screen DS when height and weight are correctly inputted. The icon IC is generated as a diagram simulating shape of human body. When height and weight are correctly entered, the icon IC, whose shape is similar to human body shape with balanced height and weight, is displayed as illustrated in FIG. 9 (in the step S15).

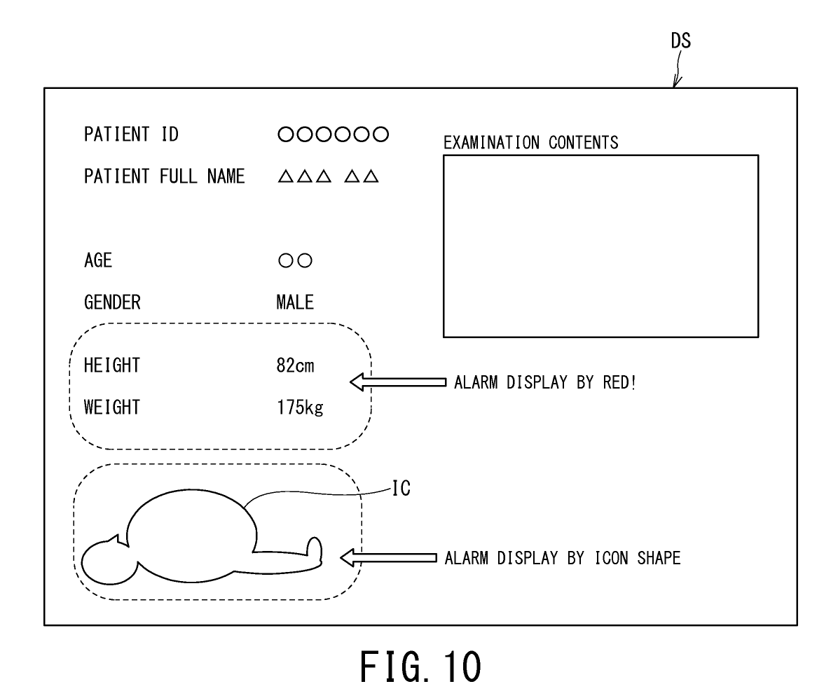

By contrast, FIG. 10 is a chart showing an example of the icon IC displayed on the input screen DS when height and weight are erroneously inputted. In FIG. 10, the value of height of the patient is erroneously entered as a weight value and the value of weight of the patient is erroneously entered as a height value like FIG. 8, and the BMI becomes an abnormally large value. Thus, height and weight are displayed by red as the alarm. In addition to this alarm display, the icon IC whose shape is transformed in accordance with BMI is further displayed (in the step S16).

Although shape of the transformed icon is not limited to a special one, shape which can immediately draw attention of an operator is desirable. For example, when BMI is calculated as an abnormally large value, attention of an operator can be immediately drawn to the erroneously inputted values by displaying the icon IC whose shape is obtained by exaggeratingly enlarging the size of abdominal part with respect to normal human body shape as shown in FIG. 10.

As explained above, according to the magnetic resonance imaging apparatus 1 and the medical information processing device 501 of the present embodiment, erroneous input of height and weight can be infallibly prevented by an easy method.

Incidentally, the term “processor” used for explaining the processing circuitry 40 and 509 means, for instance, a circuit such as a CPU (Central Processing Unit), a GPU (Graphics Processing Unit), an ASIC (Application Specific Integrated Circuit), a programmable logic device including an SPLD (Simple Programmable Logic Device) and a CPLD (Complex Programmable Logic Device) as examples, an FPGA (Field Programmable Gate Array), and so on. A processor implements various types of functions by reading out programs stored in a storage circuit and executing the programs.

The number of processors provided for each of the processing circuitry 40 and 509 may be one, two, or more.

When plural processors are included in each of the processing circuitry 40 and 509, a memory for storing programs may be provided for each processor or one memory may collectively store all the programs corresponding to the functions of each processor.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. A magnetic resonance imaging apparatus comprising:
an input device by which patient information including at least height and weight is inputted;
a display; and
processing circuitry configured to
calculate a BMI (Body Mass Index) indicated by weight (kg)/{height (m)}² based on the height and weight,
determine whether the BMI is within a range of a predetermined threshold value or not; and
cause the display to display an alarm when the BMI is not within the range of a predetermined threshold value.

2. The magnetic resonance imaging apparatus according to claim 1,
wherein the processing circuitry is configured to cause the display to display information to prompt confirmation of at least one of inputted height information and inputted weight information, as the alarm.

3. The magnetic resonance imaging apparatus according to claim 1,
wherein the processing circuitry is configured to cause the display to display the alarm by displaying characters indicative of the height and weight, in such a manner that at least one of color of the characters, size of the characters, and thickness of the characters is changed from a display aspect when the BMI is within the range of a predetermined threshold value.

4. The magnetic resonance imaging apparatus according to claim 3,
wherein the input device is configured so that at least one of the predetermined threshold value, the color of the characters, the size of the characters, and the thickness of the characters inputted via the input device is changeable by a user's manipulation.

5. The magnetic resonance imaging apparatus according to claim 1,
wherein the processing circuitry is configured to
further determine degree of deviation between the BMI and the range of a predetermined threshold value; and
cause the display to display the alarm in a multistage manner by changing a display aspect of the alarm in accordance with the degree of deviation.

6. The magnetic resonance imaging apparatus according to claim 5,
wherein the processing circuitry is configured to cause the display to display values of the height and weight inputted via the input device in a multistage manner by using a plurality of mutually different colors in accordance with the degree of deviation.

7. The magnetic resonance imaging apparatus according to claim 6,
wherein the input device is configured to receive input to change at least one of a threshold value used for determining the degree of deviation and range of the plurality of mutually different colors.

8. The magnetic resonance imaging apparatus according to claim 1,
wherein the processing circuitry is configured to cause the display to
(a) further display an icon similar to human body shape, and
(b) display the icon whose shape is transformed based on the BMI, when the BMI is out of the range of a predetermined threshold value.

9. The magnetic resonance imaging apparatus according to claim 2,
wherein the processing circuitry is configured to cause the display to
(a) further display an icon similar to human body shape, and
(b) display the icon whose shape is transformed based on the BMI, when the BMI is out of the range of a predetermined threshold value.

10. The magnetic resonance imaging apparatus according to claim 3,
wherein the processing circuitry is configured to cause the display to
(a) further display an icon similar to human body shape, and
(b) display the icon whose shape is transformed based on the BMI, when the BMI is out of the range of a predetermined threshold value.

11. The magnetic resonance imaging apparatus according to claim 5,
wherein the processing circuitry is configured to cause the display to
(a) further display an icon similar to human body shape, and
(b) display the icon whose shape is transformed based on the BMI, when the BMI is out of the range of a predetermined threshold value.

12. A medical information processing device comprising:
an input device by which patient information including at least height and weight is inputted;
a display; and
processing circuitry configured to
calculate a BMI (Body Mass Index) indicated by weight (kg)/{height (m)}² based on the height and weight,
determine whether the BMI is within a range of a predetermined threshold value or not; and
cause the display to display an alarm when the BMI is not within the range of a predetermined threshold value.

13. The medical information processing device according to claim 12,
wherein the processing circuitry is configured to cause the display to display information to prompt confirmation of at least one of inputted height information and inputted weight information, as the alarm.

14. The medical information processing device according to claim 12,
wherein the processing circuitry is configured to cause the display to display the alarm by displaying characters indicative of the height and weight, in such a manner that at least one of color of the characters, size of the characters, and thickness of the characters is changed from a display aspect when the BMI is within the range of a predetermined threshold value.

15. The medical information processing device according to claim 12,
wherein the processing circuitry is configured to
further determine degree of deviation between the BMI and the range of a predetermined threshold value; and
cause the display to display the alarm in a multistage manner by changing a display aspect of the alarm in accordance with the degree of deviation.

16. A patient information display method comprising:
acquiring patient information including at least height and weight;
calculating a BMI (Body Mass Index) indicated by weight (kg)/{height (m)}² based on the height and weight;
determining whether the BMI is within a range of a predetermined threshold value or not; and
displaying an alarm when the BMI is not within the range of a predetermined threshold value.

17. The patient information display method according to claim 16,
wherein the alarm is display of prompting confirmation of at least one of inputted height information and inputted weight information.

18. The patient information display method according to claim 16,
wherein the alarm is performed by changing at least one of color of characters indicative of the height and weight, size of the characters, and thickness of the characters, from a display aspect when the BMI is within the range of a predetermined threshold value.

19. The patient information display method according to claim 16, further comprising determining degree of deviation between the BMI and the range of a predetermined threshold value,
wherein the alarm is performed in a multistage manner by changing a display aspect of the alarm in accordance with the degree of deviation.
